(19) United States
(12) Patent Application Publication
(10) Pub. No.: US 2026/0060908 A1
(43) Pub. Date: Mar. 5, 2026

(54) USE OF FATTY ACIDS FOR MICROBIOME BENEFIT AND COMPOSITIONS COMPRISING THE SAME

(71) Applicant: Conopco, Inc., d/b/a UNILEVER, Hoboken, NJ (US)

(72) Inventors: Anindya DASGUPTA, Bangalore (IN); Srikala KUMARAN, Bangalore (IN)

(73) Assignee: Conopco, Inc., d/b/a UNILEVER, Hoboken, NJ (US)

(21) Appl. No.: 19/101,606

(22) PCT Filed: Jul. 3, 2023
(86) PCT No.: PCT/EP2023/068163

§ 371 Date: Feb. 6, 2025

(30) Foreign Application Priority Data
Aug. 8, 2022 (EP) 22189333.2

(51) Int. Cl. A61K 8/36; A61K 8/34; A61Q 17/00; A61Q 19/10

(52) U.S. Cl. CPC A61K 8/361; A61K 8/345; A61Q 17/00; A61Q 19/10; A61K 2800/5922; A61K 2800/596

(57) ABSTRACT

The present invention relates to use of specific group of fatty acids and compositions comprising them for delivering microbiome benefit to skin. This benefit ensures that the skin is protected against undesirable bacteria. The present invention delivers the benefit by the selective fatty acids i.e one having C18 carbons atoms or more. preferably one having C20 carbon atoms or more acting as prebiotics for skin commensal bacteria like S. epidermidis and S Hominis. The efficacy is improved by combining fatty acids with poly hydric alcohol like glycerol and/or by combining with fatty acid esters like glycerol monostearate. Combination of certain unsaturated fatty acids is also seen to interact synergistically to provide this benefit.

### FIELD OF THE INVENTION

The present invention relates to use of specific group of fatty acids and compositions comprising them for delivering microbiome benefit to skin. This benefit ensures that the skin is protected against undesirable bacteria. The present invention delivers the benefit by selective fatty acids acting as prebiotics for skin commensal bacteria like S. epidermidis and S hominis.

### BACKGROUND OF THE INVENTION

Skin in mammals are considered as one of the largest organs in the body, especially one which has the largest surface area. Skin forms the first line of defense against microorganisms which may invade the body though the air, water, food or material that come in contact with the body. When the body is infected on the skin or systemically, traditional approach to such hygiene problems has been to treat the skin/body with antimicrobial actives that reduce or kill the germs. Recent research indicates that a lot of the bacteria that permanently reside on the skin (called skin commensal bacteria) do not actually cause infections, rather they are beneficial bacteria that protect the skin against disease causing pathogens. Several mechanisms have been proposed to explain the protection and some of the popular ones are: physically occupying space on skin to prevent colonization of pathogens; producing metabolites that ward off harmful possibly pathogenic organisms; generating metabolites that strengthen the innate defense mechanisms to prevent infection by harmful pathogens; and providing other benefits such as maintaining skin pH, barrier function etc. Thus, of late, there is a trend in moving away from the approach of treating the skin with broad spectrum antimicrobial actives to kill all microorganisms present on skin (or any other part of the body) as a means of treating infections. Rather, the approach is more towards targeted or selective inhibition/killing of the desired microorganism to the exclusion of the skin commensal organism. This ensures that the skin microbiome is maintained in a healthy balanced state for long term hygiene and health.

The present inventors, in looking to solve the problem of selective kill of non-commensal bacteria on skin like E. coli and S. aureus directed their research to boosting the number and functionality of skin commensal bacteria through use of prebiotics or Selective Fermentation Inducers (SFI) They found, during the course of the research, that the population of skin commensal bacteria like S. epidermidis and S. hominis can be well maintained or further grown on skin through use of select fatty acid viz. C18 or higher preferably C20 or higher to the exclusion of the lower molecular weight fatty acids. This efficacy can be further enhanced through use of agents like glycerol and/or glycerol monostearate when combined with C16 or higher fatty acid. To the knowledge of the present inventors such select compounds, combination of compounds or personal care composition comprising them is not known for delivering this benefit. WO20216757 (Universiteit Gent) discloses acids which steer the microbiome by inhibiting the growth of specific malodour producing bacteria and/or promoting the growth of bacteria known to be beneficial in relation to malodour. It does not specifically disclose that fatty acid whether it be saturated or unsaturated C18+ fatty acid, preferably C20+ fatty acid to the exclusion of lower molecular weight fatty acid is especially good for delivering this benefit.

It is thus an object of the present invention to provide for compounds or a composition comprising them for use in promoting microbiome balance to skin.

### SUMMARY OF THE INVENTION

The first aspect of the present invention relates to use of C18 to C24 fatty acid, preferably C20 to C24 fatty acid for microbiome benefit to skin.

A preferred aspect of the present invention relates to use of a personal care composition comprising (i) C18 to C24 fatty acid preferably C20 to C24 fatty acid and (ii) a cosmetically acceptable carrier; for microbiome benefit to skin.

Another aspect of the present invention relates to a composition comprising
(i) 0.2% to 10% wt % C20 to C24 fatty acid; and
(ii) a cosmetically acceptable carrier;
   wherein the composition comprises less than 1 wt %, preferably less than 0.1 wt % C10 to C18 fatty acid.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

“Skin” as used herein is meant to include skin on any part of the body (e.g. face, neck, chest, back, arms, underarms, hands, legs, buttocks and scalp). It is especially useful for protecting the skin of babies. By babies is meant a child whose age is less than five years preferably less than three years more preferably less than a year. Such use against pathogens is preferably non-therapeutic, for example, delivered through a cosmetic or personal care composition. Further the composition of the invention and the method of the invention are also preferably for non-therapeutic use.

Any preference described hereinbelow with regard to one aspect of the invention (e.g. the composition or the use according to the invention) is also preferred for use in any one of the other aspects of the invention.

The present invention relates to use of C18 to C24 fatty acid, preferably C20 to C24 fatty acid for microbiome benefit to skin. By way of this benefit, the skin is protected against non-commensal bacteria via nourishing skin commensal bacteria. Further, the present invention relates to use of this active in a personal care composition preferably a skin care composition also comprising a cosmetically acceptable carrier, for microbiome benefit. Further, it relates to use of the skin care composition as commensal bacteria nourishing agent. Alternatively, the present invention also relates to use of the active and the personal care composition as prebiotic for commensal bacterium. The chosen fatty acids could be saturated or unsaturated, preferably saturated. Suitable saturated fatty acids could be C18:0 (stearic acid), C20:0 (arachidic acid), or C24:0 (lignoceric acid). Examples of unsaturated fatty acids could be C18:1 (Oleic acid), C18:2 (linoleic acid) or C18:3 (linolenic acid). It is particularly preferred that the benefits of this invention may be obtained by a fatty acid mixture of oleic acid and linoleic acid. Thus, a preferred aspect of the present invention relates to use of the mixture of oleic acid and linoleic acid for microbiome benefits.

The microbiome benefit as per this invention is achieved by way of enhancing growth of skin commensal bacteria like S. epidermidis and S. Hominis.

Another aspect of the present invention thus, relates to use of a personal care composition comprising (i) C18 to C24 fatty acid preferably C20 to C24 fatty acid and (ii) a cosmetically acceptable carrier; for microbiome benefit to skin. This is also achieved by way of enhancing growth of skin commensal bacteria like S. epidermidis and S. hominis. The composition as per this invention preferably comprises 0.2% to 10 wt % C18 to C24 fatty acid. An especially preferred aspect relates to use of the composition which comprises 0.2 to 10 wt % of C20 to C24 fatty acids. A preferred aspect of the present invention relates to use of the composition which comprises less than 1 wt % C10 to C16 fatty acid, preferably less than 0.1 wt % C10 to C16 fatty acid for microbiome benefit. An even more preferred aspect of the invention relates to the use of the composition comprising less than 1 wt % C10 to C18 fatty acid, preferably less than 0.1 wt % C10 to C18 fatty acid, for microbiome benefit. Yet another preferred aspect of the present invention relates to use of a personal care composition comprising (i) C18 to C24 fatty acid comprising a mixture of oleic acid and linoleic acid and (ii) a cosmetically acceptable carrier; for microbiome benefit to skin.

Another aspect of the present invention relates to a composition comprising
(i) 0.2% to 10% wt % C20 to C24 fatty acid; and
(ii) a cosmetically acceptable carrier;
   wherein the composition comprises less than 1 wt %, preferably less than 0.1 wt % C10 to C18 fatty acid.

Yet another aspect of the present invention relates to a composition comprising
(i) 0.2% to 10% wt % C16 to C24 fatty acid;
(ii) a polyhydric alcohol and
(iii) a cosmetically acceptable carrier;
   wherein the composition comprises less than 1 wt %, preferably less than 0.1 wt % C10 to C14 fatty acid.

As per this aspect of the present invention the C16 to C24 fatty acid may preferably comprise a mixture of oleic acid and linoleic acid.

As per any aspect of the present invention involving a mixture of oleic acid and linoleic acid, the benefits may be obtained by use of a combination of oils rich in these fatty acid esters. When in use, a certain percentage say about 10% of the mixture of oils are hydrolyzed to generate the desired unsaturated fatty acids to deliver the benefits of the invention. Suitable oils to be mixed are those rich in oleic acid and linoleic acid preferably olive oil and sunflower oil.

Inclusion of polyhydric alcohol enhances the microbiome benefit by synergistically interacting with the selective fatty acids. The polyhydric alcohol may be selected from one or more of glycerol, 1,3-butylene glycol, propylene glycol, 1,3-propanediol, pentylene glycol, hexylene glycol, and sorbitol. The most preferred polyhydric alcohol for inclusion in the composition of the invention is glycerol. The composition of the invention may additionally comprise a fatty acid ester preferably glycerol monostearate.

It is to be understood that the benefits disclosed and claimed by the selective fatty acids in the present invention could also be achieved by a composition comprising the combination of the selective fatty acids (to the exclusion of other fatty acids) and a cosmetically acceptable carrier. Conversely, the benefits disclosed and claimed in the composition and method of the present invention are to be understood to be due to the inclusion of the selective fatty acids. Such a composition could be in the form of a leave-on composition. Alternatively and equally preferably it could be delivered through a wash-off format for delivering selective protective benefit to topical areas e.g. skin and/or hair of mammals, especially humans. Such a composition includes any product applied to a human body for also improving appearance, cleansing, or general aesthetics. The composition of the present invention may be delivered with a topically acceptable carrier.

The fatty acids as per the invention and composition comprising them are preferably a prebiotic for a skin commensal bacterium. The skin commensal bacteria is preferably S. epidermidis. The skin is protected as per this invention from non-commensal bacteria which may be E. coli, S. aureus, P aeruginosa, P acnes or mixtures thereof. Consumers often find such non-commensal bacteria to be unwanted or undesirable as they may provide certain negative attributes like producing itchiness, discomfort or malodour. In certain cases, they may even be harmful or in extreme cases be pathogenic. Therefore, the non-commensal bacteria are preferably unwanted bacteria, undesirable bacteria, harmful bacteria and/or pathogenic bacteria or bacteria with a combination of these attributes.

The composition of the invention is preferably a wash-off composition and this is enabled by including 0.25 to 80% by weight of a surfactant. In general, the surfactants may be chosen from the surfactants described in well known textbooks like “Surface Active Agents” Vol. 1, by Schwartz & Perry, Interscience 1949, Vol. 2 by Schwartz, Perry & Berch, Interscience 1958, and/or the current edition of “Mccutcheon's Emulsifiers and Detergents” published by Manufacturing Confectioners Company or in “Tenside-Taschenbuch”, H. Stache, 2nd Edn., Carl Hauser Verlag, 1981. Any type of surfactant, i.e. anionic, cationic, nonionic, zwitterionic or amphoteric can be used but preferred surfactant is of the anionic, amphoteric or non-ionic type, most preferably of the anionic and amphoteric type.

The pH of a wash off composition as per the present invention is in the range of 5 to 11, preferably in the range of 5.5 to 10.

The surfactant may be a soap. Soap is a suitable surfactant for personal washing applications of composition of the invention. The soap is preferably C8-C24 soap, more preferably C10-C20 soap and most preferably C12-C16 soap. The soap may or may not have one or more carbon-carbon double bond or triple bond. The cation of the soap may be alkali metal, alkaline earth metal or ammonium. Preferably, the cation of the soap is selected from sodium, potassium or ammonium. More preferably the cation of the soap is sodium or potassium.

The soap may be obtained by saponifying a fat and/or a fatty acid. The fats or oils generally used in soap manufacture may be such as tallow, tallow stearines, palm oil, palm stearines, soya bean oil, fish oil, castor oil, rice bran oil, sunflower oil, coconut oil, babassu oil, palm kernel oil, and others. In the above process the fatty acids are derived from oils/fats selected from coconut, rice bran, groundnut, tallow, palm, palm kernel, cotton seed, soyabean, castor etc.

A typical fatty acid blend consisted of 5 to 30% coconut fatty acids and 70 to 95% fatty acids ex hardened rice bran oil. Fatty acids derived from other suitable oils/fats such as groundnut, soybean, tallow, palm, palm kernel, etc. may also be used in other desired proportions. The most preferred soap is a laurate soap. The soap, when present in solid forms of the present invention is present in an amount of 30 to 90%, preferably from 50 to 85%, more preferably 55 to 75% by weight of the composition. The soap, when present in liquid forms of the composition is present in 0.5 to 20%, preferably from 1 to 10% by weight of the composition.

Alternatively the surfactants are non-ionic surfactants, such as C8-C22, preferably C8-C16 fatty alcohol ethoxylates, comprising between 1 and 8 ethylene oxide the surfactants are preferably selected from primary alkyl sulphate, secondary alkyl sulphonates, alkyl benzene sulphonates, or ethoxylated alkyl sulphates. The composition may further comprise an anionic surfactant, such as alkyl ether sulphate preferably those having between 1 and 3 ethylene oxide groups, either from natural or synthetic source and/or sulphonic acid. Especially preferred are sodium lauryl ether sulphates. Alkyl polyglucoside may also be present in the composition, preferably those having a carbon chain length between C6 and C16. Suitable surfactant concentrations in liquid forms of cleaning application are generally more than 0.5 but less than 10%, preferably from 1 to 5% by weight of the composition. In solid compositions, the surfactant is preferably present in 5 to 40%, preferably from 10 to 30% by weight of the composition.

The cleansing composition of the invention may also be delivered through a moisturizing bar or a moisturizing liquid composition. Moisturizing bar compositions comprising fatty acyl isethionates (e.g. cocyl isethionate) are especially preferred.

Fatty acyl isethionates (e.g., cocoyl isethionates) surfactant “products” are defined as mixtures of anionic acyl isethionate surfactants and fatty acids/fatty acid soaps. They are highly desirable in personal care skin or hair cleansing products, particularly in personal care products, because they lather well, are mild to the skin and have good emollient properties. Typically, fatty acid isethionate surfactant products are produced by esterification of fatty acids or by reaction of fatty acid chloride having carbon chain length of C8 to C20 with isethionate. A typical surfactant product containing fatty acyl isethionate contains about 40 to 95 wt. % acid isethionate, and 5 to 50 wt. %, typically 10 to 40 wt. % free fatty acid, in addition to isethionate salts, typically at less than 5%, and trace (less than 2 wt. %) of other additives. Fatty acid soap may be included in the range of 5 to 15 wt %. Other surfactants like betaines may be included in 1 to 5 wt %. Water is generally included in 2 to 8 wt % of the composition.

It is desirable to formulate compositions which have relatively mild surfactants. One combination of surfactants which is particularly desirable is combination of alkyl isethionate (e.g., fatty acid esters of isethionate acid such as sodium lauroyl or sodium cocoyl isethionate) and alkyl taurate (e.g., alkyl taurate amides such as N-methyl taurate). Alternatively, the compositions could also be formulated with taurates alone in the absence of isthionates. Surfactant mixtures have the advantage that they are sulfate-free. Further, they offer the ability to formulate isotropic systems at neutral and slightly acidic pH. Thus, they allow the use of more skin friendly preservation systems. It is also desirable to provide compositions which have high levels of glycerol (e.g., 20% or more, preferably 25% to 60%, preferably 30 to 55% by wt.) because the glycerol provides excellent sensory feel (e.g., moisturization feel).

Water may preferably be present in 10 to 90% by weight of the composition depending on the format of the composition. In solid composition water may be present in 10-30%, while in liquid or semi-solid composition, water may be present in 40 to 90%.

When the composition in accordance with the invention is a leave on composition, it preferably comprises one or more of surfactant, emollient, humectant, pigment and preservative. They are most preferably produced for use in the form of a cream, lotion or gel.

The pH of a leave-on composition as per the present invention is in the range of 5 to 9, preferably in the range of 5.5 to 8.

The carrier acts as diluent or dispersant for the ingredients of the compositions. The carrier may be aqueous-based, anhydrous or an emulsion, whereby a water-in-oil or oil-in-water emulsion is generally preferred. If the use of water is desired, water typically makes up the balance of the composition, which most preferably is from 40 to 80% by weight of the composition. In addition to water, organic solvents may optionally be included as carrier to assist any other carrier in the compositions of the present invention. Examples include alkanols like ethyl and isopropyl alcohol.

Other suitable organic solvents include ester oils like isopropyl myristate, cetyl myristate, 2-octyldodecyl myristate, avocado oil, almond oil, olive oil and neopentylglycol dicaprate. Typically, such ester oils assist in emulsifying the compositions, and an effective amount is often used to yield a stable, and most preferably, water-in-oil emulsion. Emollients may also be used, if desired, as a carrier. Alcohols like 1-hexadecanol (i.e. cetyl alcohol) are preferred. Other emollients include silicone oils and synthetic esters. Silicone oils suitable for use include cyclic or linear polydimethylsiloxanes containing from 3 to 9, preferably from 4 to 5 silicon atoms. Non-volatile silicone oils useful as emollients include polyalkyl siloxanes, polyalkylaryl siloxanes and polyether siloxane copolymers. The non-volatile polyalkyl siloxanes useful polydimethylsiloxanes. Silicone elastomers may also be used. The ester emollients that may optionally be used are:
(i) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate;
(ii) ether-esters such as fatty acid esters of ethoxylated fatty alcohols;
(iii) polyhydric alcohol esters. Ethylene glycol mono and di-fatty acid esters, diethylene glycol mono-and di-fatty acid esters, polyethylene glycol (200-6000) mono-and di-fatty acid esters, propylene glycol mono-and di-fatty acid esters, polypropylene glycol 2000 monooleate, polypropylene glycol 2000 monostearate, ethoxylated propylene glycol monostearate, glyceryl mono-and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters;
(iv) wax esters such as beeswax, spermaceti, stearyl stearate and arachidyl behenate; and,
(v) sterols esters, of which cholesterol fatty acid esters are examples.

Emollients, when present, typically make up from 0.1 to 50% by weight of the composition, including all ranges subsumed therein.

Moisturisation may be improved through use of petrolatum or paraffin. Thickeners may also be utilized as a portion of the carrier in the compositions. Typical thickeners include cross-linked acrylates (e.g. Carbopol® 982), hydrophobically-modified acrylates (e.g. Carbopol®1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.001 to 5, optimally from 0.01 to 0.5% by weight of the composition.

Surfactants may also be present. When present, the total amount of surfactants is 2 to 40% by weight, and preferably from 4 to 20% by weight, optimally from 5 to 12% by weight of the composition. The surfactant is selected from the group consisting of anionic, nonionic, cationic and amphoteric actives. Particularly preferred nonionic surfactants are those with a C₁₀₋₂₀ fatty alcohol or acid hydrophobe condensed with from 2 to 100 moles of ethylene oxide or propylene oxide per mole of hydrophobe; mono-and di-fatty acid esters of ethylene glycol; fatty acid monoglyceride; sorbitan, mono-and di- C8-C20 fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C_{8 to 20} acyl isethionates, acyl glutamates, C_{8 to 20} alkyl ether phosphates and combinations thereof.

Various other ingredients may also be used in compositions. Actives are defined as skin benefit agents other than emollients and other than ingredients that merely improve the physical characteristics of the composition. Although not limited to this category, general examples include extender pigments such as talcs and silicas, as well as alpha-hydroxy acids, beta-hydroxy acids and zinc salts.

Beta-hydroxy acids include salicylic acid. Zinc oxide and zinc pyrithione are examples of useful zinc salts.

Suitable preservatives include alkyl esters of p-hydroxybenzoic acid, hydantoin derivatives, propionate salts, and a variety of quaternary ammonium compounds. Particularly preferred preservatives are methyl paraben, propyl paraben, phenoxyethanol and benzyl alcohol. Preservatives are from 0.1 to 2% by weight of the composition.

The packaging could be a patch, bottle, tube, roll-ball applicator, propellant driven aerosol device, squeeze container or lidded jar.

Without wishing to be bound by theory the present inventors believe that select fatty acids used in the present invention work well for microbiome benefit as it provides a delicate balance between getting maximal benefit as a prebiotic while ensuring that the antimicrobial efficacy is as low as possible.

The present invention is therefore intended to increase, promote, improve, maintain or sustain skin health and skin resiliency through improving the barrier health or NMF. This is attained through ensuring a healthy skin ecosystem through balanced microbiome health. By balanced microbiome health is meant that the ratio of commensal bacteria to non-commensal bacteria is maintained in a desired range for the skin health to be maintained, over both the short time as well as extended time frames.

This invention is especially useful for use on skin of babies where the use of broad spectrum anti bacterial agents are considered too harsh as it tends to interfere with the growth and maintenance of a healthy skin microbiome for long term health of the babies as they grow into adults. It is well accepted that baby skin barrier is more vulnerable than adult skin. Baby skin barrier is more delicate and water loss from the surface is faster than in adult skin. Similarly, the microbiome of baby skin is seen to be different from that of adult skin. Soon after birth, the baby's skin as well as skin microbiome continues to change for up to about three to five years. Together with the barrier function, the microbiome plays a critical role in skin's natural defence. It is thus imperative that the skin microbiome of babies and infants should be nourished to allow for its natural growth and maturation thereby maintaining optimum skin health.

Thus, whether used on skin of babies or others, the composition for use in the present invention is substantially free of a conventional antimicrobial compound. By substantially free is meant that conventional antimicrobial actives are present in less than 0.1 wt %, more preferably less than 0.05 wt %, further more preferably less than 0.01 wt %, even more further preferably less than 0.001% by weight of the composition. By conventional antimicrobial actives is meant an antimicrobial active which kills or inhibits bacteria which attack skin like E. coli, S. aureus, P aeruginosa, or P acnes among others. Preservatives which are included in compositions for their microbial stability are excluded from the definitions of antimicrobial actives mentioned above. Preservatives are included to ensure that the compositions are stable with respect to microorganisms which may grow and degrade the compositions. On the other hand, antibacterial compounds are included in compositions to hinder the growth of microorganisms which are present on the substrate (e.g skin) on which the compositions are applied.

Conventional antimicrobial compounds are generally those from the class of biguanides, bisphenols, halophenols, oligodynamic metal compounds like those of silver or zinc, cationic antimicrobial compounds or essential oil actives. Biguanide has a general base structure which may be further derivatised e.g. chlorhexidine or polyhexamethylene biguanide (PHMB). Bisphenols include triclosan or hexachlorophene. Halophenol include chloroxylenol (PCMX). Cationic compounds are another class of antimicrobial actives e.g. benzalkonium chloride, cetyl pyridinium chloride or cetyl trimethyl ammonium bromide.

The class of low boiling alcohols (which are fast acting antimicrobials) are especially not included in any substantial amount in the composition of the invention. Ideally they are absent from such compositions. By low boiling alcohols are meant monohydric alcohols with 2 to 5 carbon atoms. When the composition of the invention is formulated for use on skin of babies, it is preferred that it is substantially free of a fragrance component. By a fragrance component is meant a molecule or a group of molecules that are compounded to form a perfume. The characteristic of such compounds is that they are volatile (to varying degrees) and offer a pleasant odour. In the context of perfumes or fragrance component, “by substantially free” is meant that that they are present in less than 0.05 wt %, more preferably less than 0.01 wt %, further more preferably less than 0.005 wt %, even more further preferably less than 0.001 % by weight of the composition.

With a goal of developing a range of products suitable for all types of skin especially for nourishing baby skin, this invention took upon an objective to examine various prebiotics for skin commensal bacterial and arrived at this unique combination of a saccharide and glycerol to attain the same. Surprisingly it was found that not only does it provide microbiome balancing benefit but does that over the short term as well as over long time frames.

The present invention thus provides for a composition for prevention of growth of non-commensal bacteria on skin. Model non-commensal bacteria which have been tested and observed to be inhibited include E. coli, S. aureus, P aeruginosa, P. acnes or mixtures thereof.

The invention is capable of caring for baby's skin. One way that the invention does this is by providing a protective barrier on skin using the body's natural microbiome and through use of prebiotics to feed the natural microbiome. This way, the present invention develops a healthy and resilient skin and improve and build a better barrier. Another advantage of the present invention is that it does not disrupt the skin pH, which plays an important role in microbiome health, and replenishes skin with essential nutrients to help skin retain its natural moisture.

Though the use according to the invention generally is of a non-therapeutic nature, the composition may also be used in therapeutic applications. Thus, the composition preferably is a composition for use in therapeutically protecting skin from harmful bacteria via nourishing the skin commensal bacteria. In such cases, use in the treatment of skin conditions like eczema, atopic dermatitis, seborrheic dermatitis, acne, and dandruff.

The invention thus provides for nourishing the good microbes thus maintaining the health of the microbiome. The invention helps in maintaining the host microenvironment to help the right microbes flourish. It does this by maintaining the pH mantle of the host. Alternatively, the present invention ensures that the microbiome balancing is obtained by selectively reducing the number of harm causing microbes residing on the external surface like skin whilst helping the beneficial microbes thrive.

The invention will now be demonstrated with the help of the following non-limiting examples.

### EXAMPLES

### A-C and 1-3: Effect of Inclusion of Specific Saturated Fatty Acids on Growth of S. Epidermidis.

Experiments were carried out on the growth of S. Epidermidis in the presence of various fatty acids using the following procedure.

### Fatty Acid Stock Preparation

5% of fatty acid stock is made in DMSO-0.5 g in 10 ml of DMSO
2.68% of fatty acid (Total volume 5 ml) is made with 2.68 ml of 5% fatty acid stock+1.89 ml of DMSO+0.45 ml of Tween 80.
Test samples: 50 μl 2.68% stock in 4.95 ml 1/10^{th} diluted TSB (test samples)

### Assay Protocol

### Day1

The microorganism (S. epidermidis or S. hominis as the case may be) was streaked on TSA agar plate and incubated at 37° C. for 24 hr

### Day2

Plate cultures of the microorganism was used to set up 0.3 OD620nm (corresponding to log 108 CFU/ml) in 1× PBS (pH7.4)

The test samples (200 μl) were added to 96 well plate, followed by addition of above set cultures (20 μl).

The 96 well plate was placed in a Tecan reader and absorbance was read at 620 nm for a period of 24-30 h with a kinetic interval of 1 h

The values obtained is plotted as a growth curve with X-axis representing time in hour and Y-axis as absorbance read at 620 nm.

The data on the % increase over control at the end of 24 to 30 hours is shown in the table—1 below.

TABLE 1

| | | % increase |
|---|---|---|
| Example | Fatty acid | over control |
| Control | | 100.0 |
| A | 0.0134% Lauric acid (C12:0) | 81.8 |
| B | 0.003% myristic acid (C14:0) | 78.5 |
| C | 0.0134% palmitic acid (C16:0) | 77.4 |
| 1 | 0.0268% stearic acid (C18:0) | 161.0 |
| 2 | 0.0268% arachidic acid (C20:0) | 118.0 |
| 3 | 0.0268% lignoceric acid (C24:0) | 142.0 |

The data in the table-1 above indicates that saturated fatty acid with chain length C18 and above provides for increased growth of skin commensal bacteria S. Epiderimidis compared to control while saturated fatty acid with chain length C16 and below caused reduction in growth as compared to control.

### Examples D-F and 4-6: Effect of Inclusion of Specific Saturated Fatty Acids on Growth of S. Hominis

Experiments similar to those in Table-1 were carried out except that the microorganism used was S. Hominis. The data on growth of the S. Hominis with respect to control is shown in Table-2.

TABLE 2

| | | % increase |
|---|---|---|
| Example | Fatty acid | over control |
| Control | | 100.0 |
| D | 0.0134% Lauric acid (C12:0) | 63.0 |
| E | 0.001% myristic acid (C14:0) | 52.0 |
| F | 0.0134% palmitic acid (C16:0) | 104.0 |
| 4 | 0.0268% stearic acid (C18:0) | 154.0 |
| 5 | 0.0134% arachidic acid (C20:0) | 123.0 |
| 6 | 0.0134% lignoceric acid (C24:0) | 153.0 |

The data in table-2 above indicates the same trend for S. Hominis as observed for S. epiderimidis i.e that fatty acid with chain length C18 and above provides for increased growth of skin commensal bacteria compared to control while fatty acid with chain length C16 and below caused reduction in growth as compared to control.

### Example G, 7-8: Effect of Inclusion of Specific Unsaturated Fatty Acids on Growth of S. Epidermidis

Experiments similar to those in Table-1 were carried out except that unsaturated fatty acids as shown in Table-3 were used. The data on growth of the S. Epidermidis with respect to control is shown in Table-3.

TABLE 3

| | | % increase |
|---|---|---|
| Example | Fatty acid | over control |
| Control | | 100.0 |
| G | 0.001% Palmitoleic acid(C16:1) | 98.4 |
| 7 | 0.001% Oleic acid (C18:1) | 132.0 |
| 8 | 0.001% Linoleic acid (C18:2) | 146.2 |

The data in the table-3 above indicates that unsaturated fatty acid behaves very similar to saturated fatty acids with regard to growth of S. Epidermidis compared to control.

### Examples H.I. 9, 10: Effect of Combination of Glycerol and Fatty Acid on Growth of S. Epidermidis

Experiments similar to those in Table-1 were carried out except that a combination of glycerol and saturated fatty acid was used. The data is summarised in Table-4.

TABLE 4

| | | % increase |
|---|---|---|
| Example | Fatty acid | over control |
| Control | | 100.0 |
| H | 0.02% Glycerol | 164.0 |
| 1 | 0.0268% Stearic acid (C18:0) | 161.0 |
| 9 | 0.02% glycerol + 0.0268% stearic acid | 227.0 |
| I | 0.02% Glycerol | 149.8 |
| C | 0.0134% palmitic acid (C16:0) | 77.4 |
| 10 | 0.02% glycerol + 0.0135% palmitic acid | 167.1 |

The data in Table-4 above indicates that combining glycerol with a fatty acid higher C16 and higher provides for vastly enhanced increase in growth of S. Epidermidis as compared to control.

### Examples 11-13: Effect of a Combination of Oleic Acid and Linoleic Acid on Growth of S. Epidermidis

Experiments similar to those in Table-1 were carried out except that unsaturated fatty acids as shown in Table-3 were used. The data on growth of the S. Epidermidis with respect to control is shown in Table-5.

TABLE 5

| | | % increase |
|---|---|---|
| Example | Fatty acid | over control |
| Control | | 100 |
| 11 | 0.01% Oleic acid (C18:1) | 135 |
| 12 | 0.01% Linoleic acid (C18:2) | 132 |
| 13 | 0.01% Oleic acid + 0.01% Linoleic acid | 212 |

The data in the table-5 above indicates that a mixture of the two unsaturated fatty acid provides for synergistic benefit with regard to growth of S. epidermidis.

1.-8. (canceled)

9. A composition comprising:
(i) 0.2 to 10 wt % C20 to C24 fatty acid,
(ii) 0.25 to 80 wt % of a surfactant; and
(iii) a cosmetically acceptable carrier,
wherein the composition comprises less than 1 wt %, of a C10 to C18 fatty acid.

10. A composition comprising:
(i) 0.2 to 10 wt % C16 to C18 fatty acid,
(ii) a polyhydric alcohol;
(iii) 0.25 to 80 wt % of a surfactant; and
(iv) a cosmetically acceptable carrier;
wherein the composition comprises less than 1 wt %, of a C10 to C14 fatty acid.

11. The composition as claimed in claim 10, wherein the C16 to C18 fatty acid comprises a mixture of oleic acid and linoleic acid.

12. The composition as claimed in claim 9, wherein the composition is a leave-on product in the form of a cream, lotion, or gel.

13. The composition as claimed in claim 9, wherein the surfactant is selected from one or more of a synthetic anionic or amphoteric surfactant.

14. The composition as claimed in claim 9 further comprising a fatty acid ester.

15. The composition as claimed in claim 9, wherein the composition comprises less than 0.1 wt % of the C10 to C18 fatty acid.

16. The composition as claimed in claim 9, wherein the composition is a wash-off product.

17. The composition as claimed in claim 16, wherein the surfactant comprises alkyl taurate.

18. The composition as claimed in claim 10, wherein the polyhydric alcohol is glycerol.

19. The composition as claimed in claim 10, wherein the composition comprises less than 0.1 wt % of the C10 to C14 fatty acid.

20. The composition as claimed in claim 10, wherein the composition is a leave-on product in the form of a cream, lotion, or gel.

21. The composition as claimed in claim 10, wherein the composition is a wash-off product.

22. The composition as claimed in claim 21, wherein the surfactant comprises alkyl taurate.

23. The composition as claimed in claim 10, wherein the surfactant is selected from one or more of a synthetic anionic or amphoteric surfactant.

24. The composition as claimed in claim 14, wherein the fatty acid ester is glycerol monostearate.

25. The composition as claimed in claim 10 further comprising a fatty acid ester.

26. The composition as claimed in claim 25, wherein the fatty acid ester is glycerol monostearate.
